(19) United States
(12) Patent Application Publication
(10) Pub. No.: US 2016/0051136 A1
(43) Pub. Date: Feb. 25, 2016

(54) RETRACTING CANNULA WITH ILLUMINATION AND METHODS OF USE

(71) Applicant: Kyphon SARL, Neuchatel (CH)

(72) Inventor: Craig E. Lauchner, Mountain View, CA (US)

(21) Appl. No.: 14/462,684

(22) Filed: Aug. 19, 2014

(51) Int. Cl. A61B 1/32

(52) U.S. Cl. CPC A61B 1/32

(57) ABSTRACT

A surgical instrument includes a first member including a body having a first portion including a first extension defining a first longitudinal axis. The body includes a second portion having a projection defining a second longitudinal axis extending transverse to the first longitudinal axis. The projection includes a flange. A second member includes a first portion having a second extension and a second portion including an opening. The projection extends through the opening. The second member is movable between a first configuration in which the flange is positioned within the opening and the second extension extends parallel to the first longitudinal axis and a second configuration in which the flange is positioned outside of the opening and the second extension extends transverse to the first longitudinal axis. Systems and methods are disclosed.

### TECHNICAL FIELD

The present disclosure generally relates to medical devices for the treatment of musculoskeletal disorders, and more particularly to a surgical system and method for accessing a surgical site to facilitate treatment.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Surgical retractors may be employed during a surgical treatment to provide access and visualization of a surgical site. Such retractors space apart and support tissue and/or other anatomical structures to expose anatomical structures adjacent the surgical site and/or provide a surgical pathway to the surgical site. This disclosure describes an improvement over these prior art technologies.

### SUMMARY

In one embodiment, a surgical instrument is provided. The surgical instrument includes a first member comprising a body including a first portion comprising a first extension defining a first longitudinal axis. The body comprises a second portion including a projection defining a second longitudinal axis extending transverse to the first longitudinal axis. The projection comprises a flange. A second member comprises a first portion including a second extension and a second portion comprising an opening. The projection extends through the opening. The second member is movable between a first configuration in which the flange is positioned within the opening and the second extension extends parallel to the first longitudinal axis and a second configuration in which the flange is positioned outside of the opening and the second extension extends transverse to the first longitudinal axis. In some embodiments, systems and methods are provided.

### BRIEF DESCRIPTION OF THE DRAWINGS

The present disclosure will become more readily apparent from the specific description accompanied by the following drawings, in which:

FIG. 1 is a side, perspective view of components of one embodiment of a surgical system in accordance with the principles of the present disclosure;

FIG. 2 is a side, perspective view of components shown in FIG. 1, in part phantom;

FIG. 3 is a bottom, perspective view of components shown in FIG. 1;

FIG. 3A is a top, perspective view of components shown in FIG. 1;

FIG. 4 is a side, perspective view of components shown in FIG. 1;

FIG. 4A is a side, perspective view of components shown in FIG. 1;

FIG. 5 is a top, perspective view of components shown in FIG. 1;

FIG. 6 is a top view of components shown in FIG. 1;

FIG. 7 is a top, perspective view of components shown in FIG. 1;

FIG. 8 is a top, perspective view of a component shown in FIG. 1;

FIG. 8A is a top, perspective view of a component shown in FIG. 1;

FIG. 9 is a bottom view of a component shown in FIG. 1;

FIG. 10 is a side, perspective view of a component shown in FIG. 1;

FIG. 11 is a top, perspective view of a component shown in FIG. 1; and

FIG. 12 is a top, perspective view of a component shown in FIG. 1.

### DETAILED DESCRIPTION

The exemplary embodiments of a surgical system and related methods of use disclosed are discussed in terms of medical devices for the treatment of musculoskeletal disorders and more particularly, in terms of a surgical system and method for accessing a surgical site to facilitate treatment. In one embodiment, the surgical system includes a surgical instrument, such as, for example, a cannula that reduces costs and provides unique features that address unmet needs. In some embodiments, the surgical instrument includes components made from molded plastic.

In some embodiments, the surgical instrument includes one or a plurality of light sources, such as, for example, light emitting diodes (LEDs) for maximizing visualization of target tissues through a small incision, eliminating the need for expensive microscopes for surgery. In some embodiments, the plurality of light sources illuminate a patient's anatomy, thereby increasing effective visualization. In some embodiments, the surgical instrument includes one or a plurality of light sources between proximal and distal ends of the surgical instrument. In some embodiments, the surgical instrument includes light sources, such as, for example, light pipes for increasing effective visualization. In some embodiments, the surgical instrument includes a power source, such as, for example, a battery to provide power to a light source, for example. In some embodiments, the battery is a standard small disposable or rechargeable battery, such as, for example, a watch battery. In some embodiments, the battery is disposed in a handle of the surgical instrument. In some embodiments, the surgical instrument includes a push clip to deploy a retractable cannula. In some embodiments, the surgical instrument is made entirely from molded plastic. In some embodiments, the surgical instrument is configured to be disposable.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The surgical system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

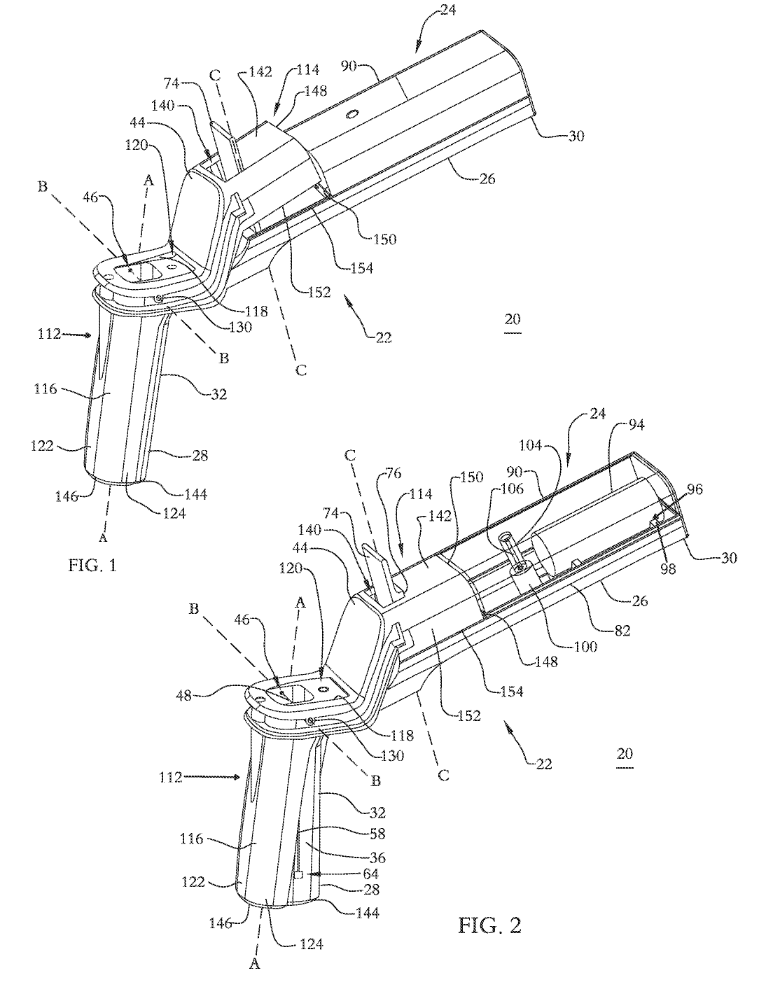

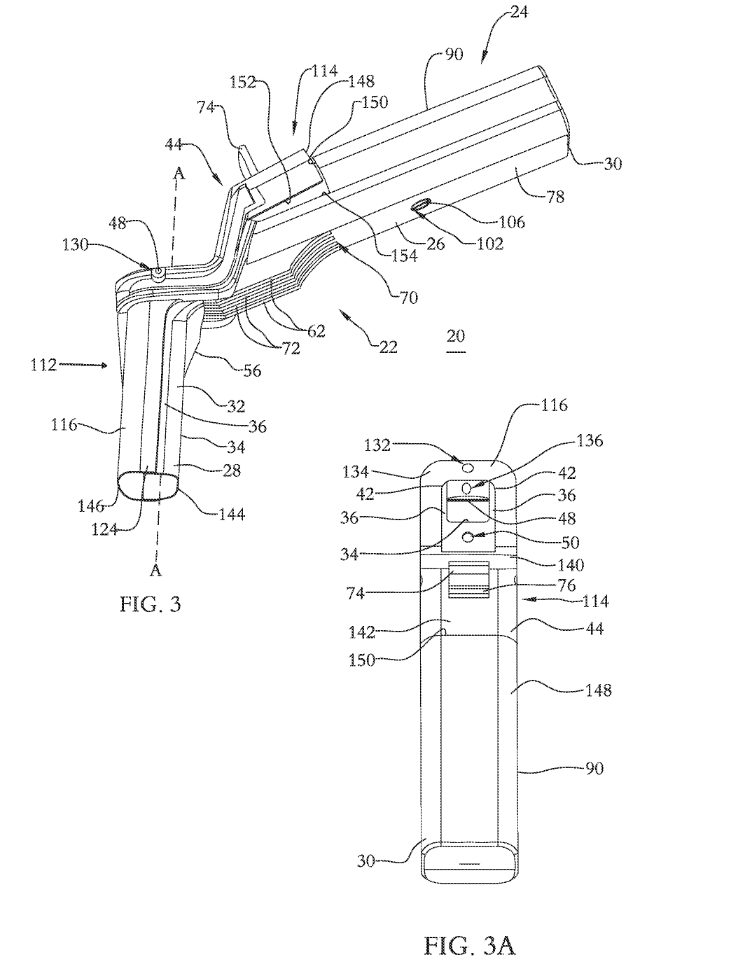

The following discussion includes a description of a surgical system and methods of employing the surgical system in accordance with the principles of the present disclosure. Alternate embodiments are also disclosed. Reference will now be made in detail to the exemplary embodiments of the present disclosure, which are illustrated in the accompanying figures. Turning to FIGS. 1-12, there are illustrated components of a surgical system 20 including a surgical instrument, such as, for example, a retractor or cannula 22, in accordance with the principles of the present disclosure.

The components of surgical system 20 can be fabricated from biologically acceptable materials suitable for medical applications, including metals, synthetic polymers, ceramics and bone material and/or their composites, depending on the particular application and/or preference of a medical practitioner. For example, the components of surgical system 20, individually or collectively, can be fabricated from materials such as stainless steel alloys, commercially pure titanium, titanium alloys, Grade 5 titanium, superelastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical system 20 may have material composites, including the above materials, to achieve various desired characteristics such as strength, rigidity, elasticity, compliance, biomechanical performance, durability and radiolucency or imaging preference. The components of surgical system 20, individually or collectively, may also be fabricated from a heterogeneous material such as a combination of two or more of the above-described materials. The components of surgical system 20 may be monolithically formed, integrally connected or include fastening elements and/or instruments, as described herein.

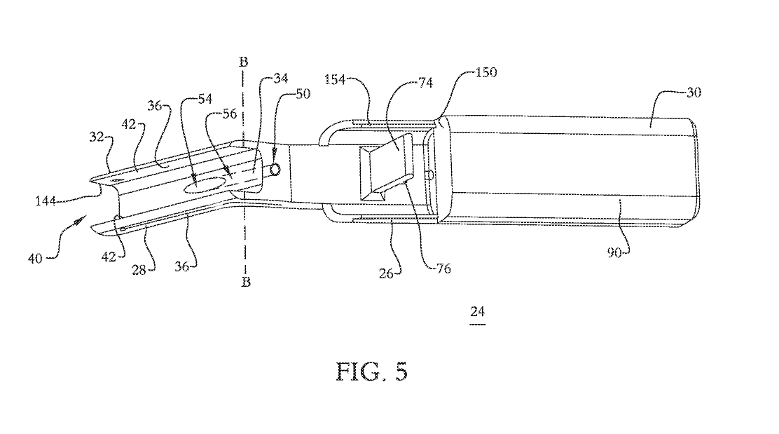

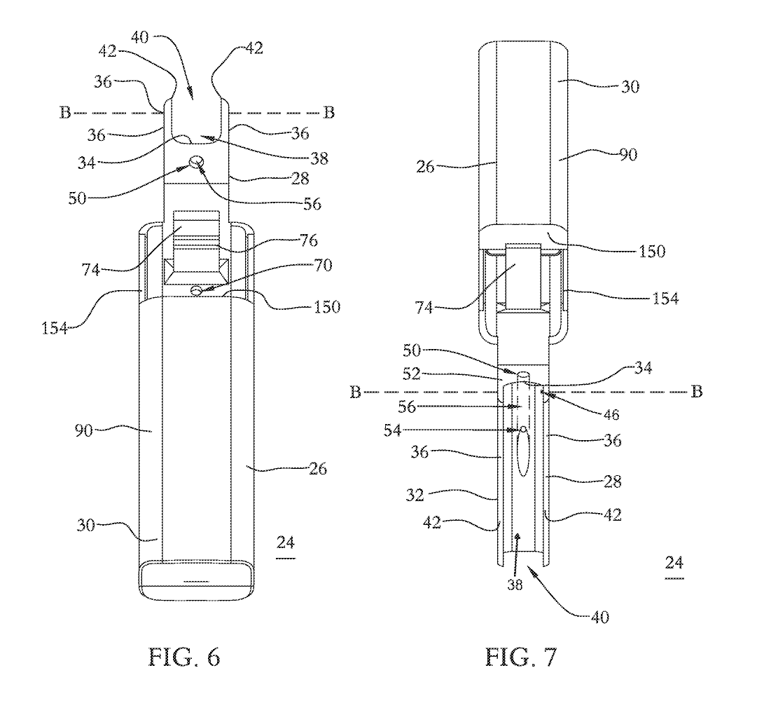

Cannula 22 includes a member 24 comprising a body 26 including a portion 28 and a portion 30. Portion 28 comprises an extension 32 defining a longitudinal axis A. Extension 32 extends away from a bottom surface of body 26. Extension 32 comprises an end wall 34 extending between opposing side walls 36, as shown in FIGS. 6 and 7, for example. Inner surfaces of walls 34, 36 define a channel 38, shown in FIG. 6, for example, extending parallel to axis A. Channel 38 comprises an opening 40 opposite wall 34, as shown in FIGS. 5-8, for example, such that channel 38 has a U-shaped cross sectional configuration perpendicular to axis A. Opening 40 extends the entire length of channel 38. Channel 38 is concavely curved at interfaces between walls 34, 36. In some embodiments, the inner surface of wall 34 is planar between the concave interfaces between walls 34, 36. In some embodiments, the inner surfaces of walls 36 are each planar extending outwardly from the concave interfaces between walls 34, 36. In some embodiments, end portions 42 of walls 36, shown in FIGS. 6-8, for example, are tapered such that portions 42 have a reduced wall thickness relative to remaining portions of walls 36 to facilitate engagement with a member 44 of cannula 22, as will be discussed. In some embodiments, end portions 42 of walls 36 are tapered such that portions 42 engage concave interfaces between walls 122, 124 of member 44, as will be discussed.

In some embodiments, channel 38 may be disposed at alternate orientations, relative to axis A, such as, for example, transverse, perpendicular and/or other angular orientations such as acute or obtuse, co-axial and/or may be offset or staggered. In some embodiments, channel 38 may have various cross section configurations, such as, for example, oval, oblong, triangular, rectangular, square, polygonal, irregular, uniform, non-uniform, variable, tubular and/or tapered. In some embodiments, the inner surface of wall 34 is concave between the concave interfaces between walls 34, 36. In some embodiments, the inner surface of wall 34 is continuously curved between the concave interfaces between walls 34, 36. In some embodiments, member 24 is made entirely of molded plastic.

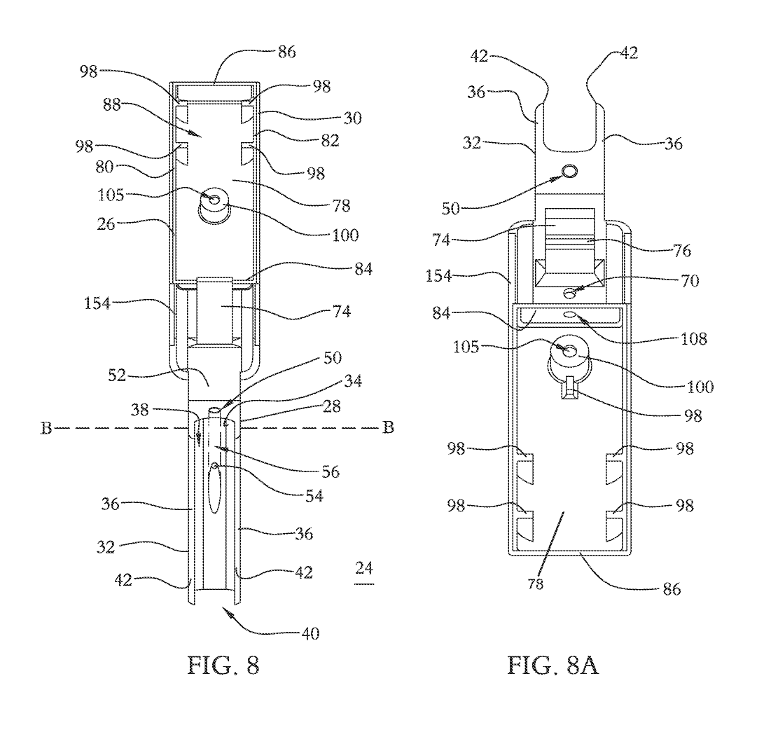

Walls 36 each include a circular opening 46 extending perpendicular to axis A through inner and outer surfaces of walls 36 such that openings 46 are aligned and/or coaxial. Openings 46 are configured for disposal of a pin 48 that couples member 44 to member 24 such that member 44 can pivot relative to member 24 about a transverse axis B defined by openings 46 extending perpendicular to axis A, as will be discussed. In some embodiments, extension 32 comprises an opening 50 in a top surface 52 of extension 32 and an opening 54 in the inner surface of wall 34, as shown in FIGS. 7 and 8, for example. Openings 50, 54 are connected by a passageway 56. Passageway 56 is configured for disposal of a surgical instrument such that the surgical instrument can be inserted into opening 50, move though passageway 56 and exit opening 54 to position the surgical instrument in channel 38.

In some embodiments, openings 46, 50, 54 may have various cross section configurations, such as, for example, oval, oblong, triangular, rectangular, square, polygonal, irregular, uniform, non-uniform, variable, tubular and/or tapered. In some embodiments, openings 46 and/or axis B may be disposed at alternate orientations, relative to axis A, such as, for example, transverse, perpendicular and/or other angular orientations such as acute or obtuse, co-axial and/or may be offset or staggered. In some embodiments, member 44 can be variously connected with member 24, such as, for example, monolithic, integral connection, frictional engagement, threaded engagement, mutual grooves, screws, adhesive, nails, barbs and/or raised element.

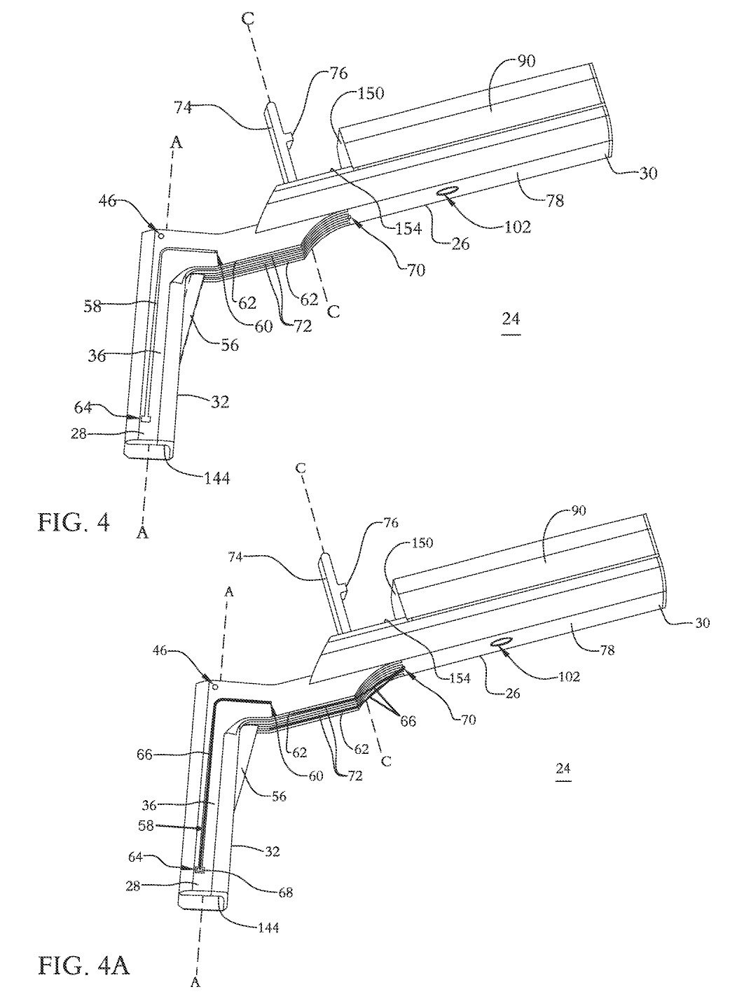

In some embodiments, body 26 includes a pair of conduits 58 each extending into an outer surface of extension 32 without extending through the inner surface of extension 32, as shown in FIGS. 4 and 4A, for example. Conduits 58 each comprise a circular aperture 60 extending through inner and outer surfaces of a respective outer ridge 62 of body 26 and a polygonal aperture 64 extending through inner and outer surfaces of a respective wall 36, as shown in FIGS. 4 and 4A, for example. Conduits 58 are each configured such that a wire 66 of a surgical instrument, such as, for example, a light source 68 can extend through an opening 70 in body 26 for positioning between ridge 62 and an inner ridge 72, as shown in FIG. 4A, for example. Wire 66 extends through aperture 60 and is positioned in conduit 58 such that light source 68 can be positioned in aperture 64 to emit light into channel 38.

In some embodiments, cannula 22 comprises a plurality of light sources 68. In some embodiments, wires 66 each comprise an anode lead and a cathode lead. In some embodiments, light sources 68 are light emitting diodes (LEDs). In some embodiments, aperture 60, aperture 64 and/or opening 70 may have various cross section configurations, such as, for example, oval, oblong, triangular, rectangular, square, polygonal, irregular, uniform, non-uniform, variable, tubular and/or tapered. In some embodiments, wires 66 and/or light sources 68 have a thickness that is less than a depth of conduit 58 such that wires 66 and/or light sources 68 may be disposed entirely in conduit 58 without protruding therefrom. In some embodiments, wires 66 and/or light sources 68 are removable. In some embodiments, wires 66 and/or light sources 68 are fixed relative to member 24. In some embodiments, wires 66 and/or light sources 68 are embedded between inner and outer surfaces of body 26, such as, for example, inner and outer surfaces of wall 36 such that wires 66 and/or light sources 68 are embedded in a wall thickness of member 24. In some embodiments, light sources 68 can be variously connected with member 24, such as, for example, monolithic, integral connection, frictional engagement, threaded engagement, mutual grooves, screws, adhesive, nails, barbs and/or raised element.

Portion 30 includes a projection 74 defining a longitudinal axis C extending transverse to axis A. Projection 74 extends away from a top surface of body 26. Projection 74 comprises a flange 76 extending perpendicular to axis C. Flange 76 faces away from portion 28 and/or extension 32. In some embodiments, axis C extends at an acute angle relative to axis A. In some embodiments, axis C extends at an angle in a range of about 15 degrees to about 75 degrees relative to axis A. In some embodiments, axis C extends at an angle in a range of about 30 degrees to about 60 degrees relative to axis A. In some embodiments, axis C extends at an angle of about 45 degrees relative to axis A. In some embodiments, projection 74 is at least somewhat movable relative to body 26 such that projection can deflect at least slightly relative to body 26 without projection 74 breaking off from body 26. In some embodiments, projection 74 comprises a rigid material such that projection 74 cannot bend relative to body 26 without projection 74 breaking off from body 26.

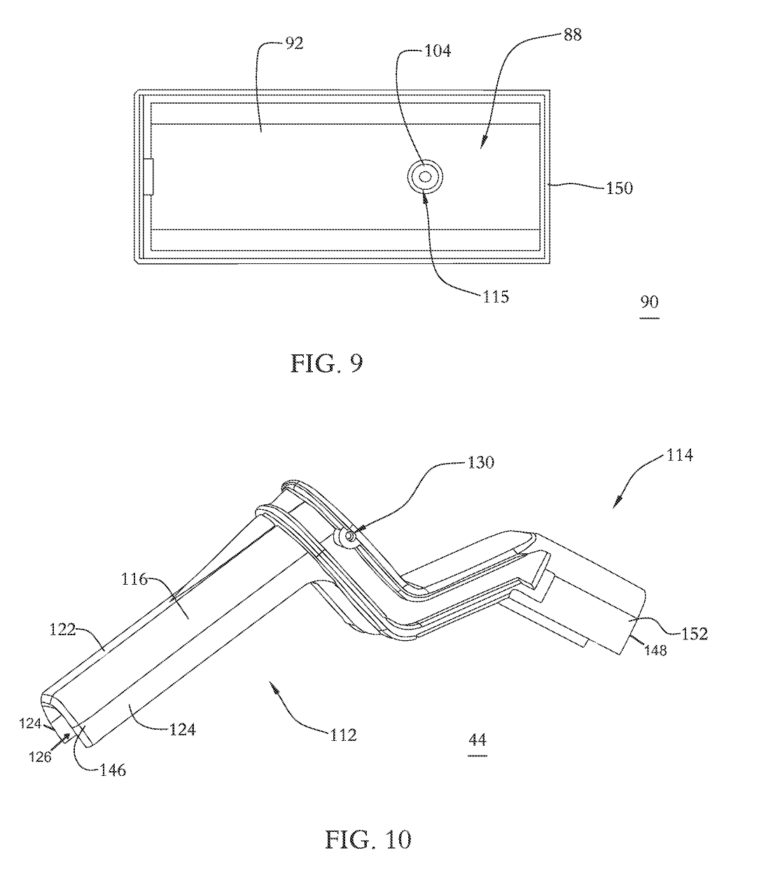

As shown in FIG. 8, for example, portion 30 includes a bottom wall 78 and opposing side walls 80, 82 extending upwardly from wall 78. Opposing end walls 84, 86 extend upwardly from wall 78. Walls 84, 86 extend between walls 80, 82. Wall 78 and inner surfaces of walls 78, 80, 82, 84 define a bottom portion of a compartment 88. Portion 30 includes a removable cover 90, shown in FIG. 9, for example, having an inner surface 92 and defining an upper portion of compartment 88. Walls 78-86 and cover 90 define a handle configured for gripping by a medical practitioner. In some embodiments, the handle includes surface configurations to enhance fixation, such as, for example, rough, arcuate, undulating, porous, semi-porous, dimpled, polished and/or textured, to facilitate gripping.

Body 26 comprises a protrusion 100 extending from wall 78 having an inner surface defining an unthreaded throughhole 105, as shown in FIGS. 8 and 8A, for example. Wall 78 comprises an opening 102, shown in FIGS. 4 and 4A, for example, extending through an outer surface of wall 78 that is in communication with throughhole 105. Cover 90 comprises a protrusion 104 extending from surface 92 having a recess 115 with a threaded inner surface, as shown in FIG. 9, for example. Walls that define a perimeter of cover 90 engage walls 80-86 such that an end surface of protrusion 100 engages an end surface of protrusion 104 and the threaded inner surface of protrusion 100 is aligned and/or coaxial with the threaded inner surface of protrusion 104. When the threaded inner surface of protrusion 100 is aligned and/or coaxial with the threaded inner surface of protrusion 104, a fastener 106 is threaded into the threaded inner surface of protrusion 100 and the threaded inner surface of protrusion 104, as shown in FIG. 2, for example, such that a threaded outer surface of fastener 106 engages the threaded inner surfaces of protrusions 100, 104 to removably fix cover 90 relative to body 26. In some embodiments, cover 90 can be variously connected with body 26, such as, for example, monolithic, integral connection, frictional engagement, threaded engagement, mutual grooves, screws, adhesive, nails, barbs and/or raised element. In some embodiments, cover 90 is made entirely of molded plastic. In some embodiments, opening 102 and/or throughhole 105 are threaded.

Compartment 88 includes a power source, such as, for example, a battery 94 removably positioned in compartment 88, as shown in FIG. 2, for example. In some embodiments, battery 94 comprises one or a plurality of grooves 96 configured for disposal of at least one rib 98 extending from wall 80 and at least one rib 98 extending from wall 82 to prevent battery 94 from moving within compartment 88, as shown in FIG. 2, for example. In some embodiments, grooves 96 are annular grooves extending about the circumference of battery 94 and are spaced apart from one another. In some embodiments, battery 94 comprises an outer surface having a cylindrical cross-sectional configuration and the outer surface of battery 94 engages ribs 98 extending from walls 80, 82 to prevent battery 94 from moving within compartment 88. In some embodiments, battery 94 is a rechargeable battery that is permanently fixed to body 26 such that battery 94 cannot be removed from compartment 88 without breaking body 26. In some embodiments, cover 90 and/or body 26 include at least one opening configured for disposal of a battery charger such that the battery charger can be inserted into the opening and plugged into battery 94 to charge battery 94. In some embodiments, the opening is one of the openings described herein. In some embodiments, battery 94 is a standard camera battery. In some embodiments, body 26 includes a rib 98 extending from protrusion 100. Battery 94 has a length defined by the distance between wall 86 and the rib 98 extending from protrusion 100 such that end surfaces of battery 94 engage wall 86 and the rib 98 extending from protrusion 100 to prevent battery 94 from moving within compartment 88.

Wires 66 are coupled to battery 94 such that wires 66 can conduct power from battery 94 to light sources 68. In some embodiments, body 26 includes a circular opening 108 extending through wall 84, as shown in FIG. 8A, for example. Opening 70 extends through wall 78, as shown in FIG. 8A, for example. This configuration allows battery 94, wires 66 and light sources 68 to be provisionally positioned in compartment 88. Wires 66 and light sources 68 may be fed through opening 108 to move light sources 68 out of compartment 88. Light sources 68 may be moved through opening 70. One light source 68 is positioned between one pair of ridges 62, 72 and one light source 68 is positioned between the other pair of ridges 62, 72. Light sources 68 are moved through apertures 60 and into conduits 58 such that light sources 68 are positioned in apertures 64 and can emit light into channel 38.

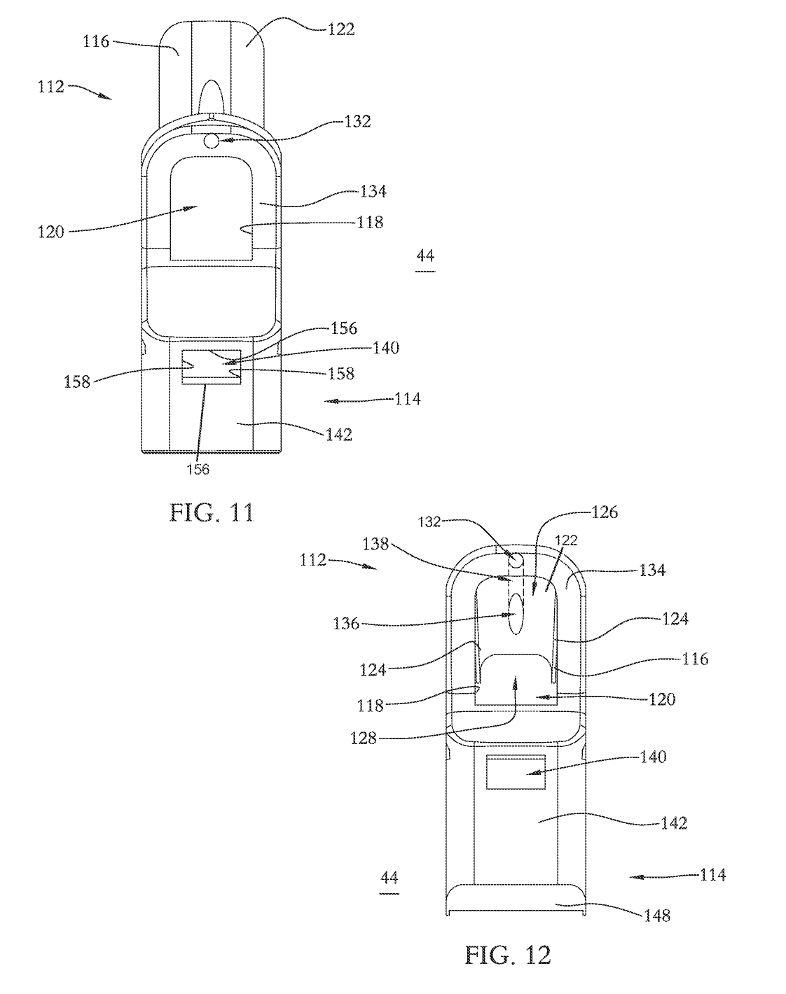

Member 44 comprises a portion 112 and a portion 114, as shown in FIG. 10, for example. Portion 112 comprises an extension 116 and an inner surface 118 defining an aperture 120, shown in FIGS. 11 and 12, for example, configured for movable disposal of at least a portion of extension 32. Extension 32 is disposed in aperture 120 such that an outer surface of extension 32 engages surface 118. Extension 116 comprises an end wall 122 extending between opposing side walls 124, as shown in FIG. 10, for example. Inner surfaces of walls 122, 124 define a channel 126. Channel 126 comprises an opening 128 opposite wall 122 such that channel 126 has a U-shaped cross sectional configuration. Opening 128 extends the entire length of channel 126. Channel 126 is concavely curved at interfaces between walls 122, 124. In some embodiments, the inner surface of wall 122 is planar between the concave interfaces between walls 122, 124. In some embodiments, the inner surfaces of walls 124 are each planar extending outwardly from the concave interfaces between walls 122, 124. In some embodiments, end portions 42 of walls 36 are tapered such that portions 42 have a reduced wall thickness relative to remaining portions of walls 36 to facilitate engagement with a member 44 of cannula 22. In some embodiments, end portions 42 of walls 36 are convexly curved and are configured to engage concavely curved interfaces between walls 122, 124 of member 44.

In some embodiments, channel 126 may have various cross section configurations, such as, for example, oval, oblong, triangular, rectangular, square, polygonal, irregular, uniform, non-uniform, variable, tubular and/or tapered. In some embodiments, the inner surface of wall 122 is concave between the concave interfaces between walls 122, 124. In some embodiments, the inner surface of wall 122 is continuously curved between the concave interfaces between walls 122, 124. In some embodiments, member 44 is made entirely of molded plastic.

Walls 124 each include a circular opening 130 extending through inner and outer surfaces of walls 124 such that openings 130 are aligned and/or coaxial. Openings 130 are aligned and/or coaxial with openings 46 such that pin 48 extends through openings 46, 130. This configuration allows member 44 to pivot relative to member 24 about pin 48 with at least a portion of extension 32 disposed within extension 116. In some embodiments, extension 116 comprises an opening 132 in a top surface 134 of extension 116 and an opening 136 in the inner surface of wall 122, as shown in FIGS. 11 and 12, for example. Openings 132, 136 are connected by a passageway 138. Passageway 138 is configured for disposal of a surgical instrument such that the surgical instrument can be inserted into opening 132, move though passageway 138 and exit opening 136 to position the surgical instrument in channel 126. In some embodiments, openings 130, 132, 136 may have various cross section configurations, such as, for example, oval, oblong, triangular, rectangular, square, polygonal, irregular, uniform, non-uniform, variable, tubular and/or tapered.

Portion 114 comprises an inner surface defining a polygonal opening 140 having projection 74 extending therethrough, as shown in FIGS. 1 and 2, for example. Opening 140 is defined by planar end surfaces 156 that each extend between planar side surfaces 158, as shown in FIG. 11, for example. Surfaces 156 extend parallel to one another and surfaces 158 extend parallel to one another. Surfaces 158 extend perpendicular to surfaces 156. Projection 74 has a width that is slightly less than the distance between surfaces 158 such that side surfaces of projection 74 engage surfaces 158 or are slightly spaced apart from surfaces 158 when projection 74 is disposed in opening 140. An inner surface of projection 74 engages one of surfaces 156 when projection 74 is disposed in opening 140.

Portion 114 is movable relative to projection 74 by pivoting member 44 relative to member 24 about axis B. This allows member 44 to pivot about the pivot point defined by pin 48 and/or axis B between a first configuration, shown in FIG. 1, in which flange 76 is positioned within opening 140 and extension 116 extends parallel to axis A and a second configuration, shown in FIG. 2, in which flange 76 is positioned outside of opening 140 and extension 116 extends transverse to axis A. Channels 38, 126 define a lumen having a maximum diameter when member 44 is in the first configuration that is less than a maximum diameter of the lumen when member 44 is in the second configuration. In some embodiments, extension 116 extends at an acute angle relative to axis A when member 44 is in the second configuration. In some embodiments, extension 116 extends at an angle in a range of about 15 degrees to about 75 degrees relative to axis A when member 44 is in the second configuration. In some embodiments, extension 116 extends at an angle in a range of about 30 degrees to about 60 degrees relative to axis A when member 44 is in the second configuration. In some embodiments, extension 116 extends at an angle of about 45 degrees relative to axis A when member 44 is in the second configuration.

In some embodiments, flange 76 engages an upper surface 142 of portion 114 when member 44 is in the second configuration and flange 76 engages a lower surface of portion 114 opposite surface 142 when member 44 is in the first configuration. The engagement of flange 76 with surface 142 and/or the lower surface of portion 114 locks member 44 in the first configuration or the second configuration. A force sufficient to move projection 74 relative to body 26 is required to move projection 74 relative to member 44 such that flange 76 disengages surface 142 or the lower surface of portion 114. Once flange 76 disengages surface 142 or the lower surface of portion 114, member 44 can be pivoted about pin 48 and/or axis B to move member 44 between the first and second configurations. In some embodiments, projection 74 is resilient such that projection 74 returns to its original position after the force is removed. In some embodiments, projection 74 is resiliently biased to engage flange 76 with surface 142 or the lower surface of portion 114.

In some embodiments, a distal end surface 144 of extension 32 is flush and/or aligned with a distal end surface 146 of extension 116 as member 44 moves between the first and second configurations, as shown in FIGS. 1 and 2, for example. Member 44 comprises an end wall 148, as shown in FIG. 12, for example. In some embodiments, wall 148 is spaced apart from an end wall 150 of cover 90 when member 44 is in the first configuration and wall 148 engages wall 150 when member 44 is in the second configuration, as shown in FIGS. 1 and 2, for example. In some embodiments, wall 148 extends parallel to wall 150 when member 44 is in the second configuration and wall 148 extends transverse to wall 150 when member 44 is in the first configuration. In some embodiments, a lower surface 152 of portion 114 is spaced apart from an upper surface 154 of portion 30 when member 44 is in the first configuration and surface 152 engages surface 154 when member 44 is in the second configuration, as shown in FIGS. 1 and 2, for example. In some embodiments, surface 152 extends transverse to surface 154 when member 44 is in the first configuration and surface 152 extends parallel to surface 154 when member 44 is in the second configuration.

In assembly, operation and use, surgical system 20, similar to that described above, is employed, for example, with a minimally invasive surgical procedure for spinal and neurosurgical applications with a patient. For example, during spine surgery, a surgeon will make an incision in the skin of a patient's back over vertebrae to be treated. One or more dilators may be employed to gradually separate the muscles and create a portal through which the surgery may be performed.

Cannula 22 is positioned adjacent the surgical site over the small incision. Cannula 22 is passed through the incision with member 44 in the first configuration. In some embodiments, cannula 22 is positioned over a dilator, such as, for example, the last dilator of a sequential dilator. Once cannula 22 is selectively positioned within the patient's anatomy, member 44 is moved from the first configuration, shown in FIG. 1, to the second configuration, shown in FIG. 2 to create a working channel defined by inner surface of extensions 32, 116. When member 44 in the second configuration, outer surfaces of extensions 32, 116 engage tissue, such as, for example, soft tissue, ligaments, tendons, cartilage and/or bone. Extensions 32, 116 space apart tissue and create access and/or a surgical pathway to a surgical site. That is, when member 44 in the second configuration, an item, such as, for example, a surgical instrument may be inserted through the working channel and/or a surgical procedure may be performed within the working channel. In some embodiments, pin 48 is removed from openings 46, 130 after member 44 is in the second configuration to provide a clear pathway for the disposal of instruments and/or to improve visualization, for example. In some embodiments, one or more surgical instruments, for example, may be inserted into the working channel through at least one of passageways 56, 138. In some embodiments, at least one imaging device, such as, for example, a camera is positioned in the working channel defined by the inner surfaces of extensions 32, 116 when member 44 is in the first configuration and/or second configuration. Once selectively positioned within the working channel, the imaging device may be used to capture images of the patient's anatomy and/or an object within the patient's anatomy. In some embodiments, the imaging is performed with at least one of light sources 68 in an on position such that light sources 68 emit light into the working channel. In some embodiments, the imaging device is inserted into the working channel through at least one of passageways 56, 138.

In some embodiments, at least one of light sources 68 are in an on position as member 44 moves from the first configuration to the second configuration. In some embodiments, at least one of light sources 68 are moved from an off position to an on position after member 44 is moved from the first configuration to the second configuration. When light sources 68 are in an on position, light sources 68 emit light into the working channel defined by inner surface of extensions 32, 116 to aid in visualization to perform a surgical procedure, for example. In some embodiments, light sources 68 are configured to emit light without creating shadows, making cannula 22 useful for imaging purposes, for example. Upon completion of the surgical procedure, cannula 22 is removed from the surgical site.

It is envisioned that the use of microsurgical and image guided technologies may be employed to access, view and repair spinal deterioration or damage, with the aid of cannula 22. It is contemplated that a surgical procedure may employ other instruments that can be mounted with cannula 22, such as, for example, nerve root retractors, tissue retractors, forceps, cutter, drills, scrapers, reamers, separators, rongeurs, taps, cauterization instruments, irrigation and/or aspiration instruments, illumination instruments and/or inserter instruments.

Cannula 22 may be employed for performing spinal surgeries, such as, for example, laminectomy, discectomy, fusion, laminotomy, nerve root retraction, foramenotomy, facetectomy, decompression, spinal nucleus or disc replacement and procedures using bone graft and implantable prosthetics including plates, rods, and bone engaging fasteners.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument, comprising:
a first member comprising a body including a first portion comprising a first extension defining a first longitudinal axis, the body comprising a second portion including a projection defining a second longitudinal axis extending transverse to the first longitudinal axis, the projection comprising a flange; and
a second member comprising a first portion including a second extension and a second portion comprising an opening, the projection extending through the opening,
wherein the second member is movable between a first configuration in which the flange is positioned within the opening and the second extension extends parallel to the first longitudinal axis and a second configuration in which the flange is positioned outside of the opening and the second extension extends transverse to the first longitudinal axis.

2. A surgical instrument as recited in claim 1, wherein the second member pivots relative to the first member to move the second member between the first and second configurations.

3. A surgical instrument as recited in claim 1, wherein a pin extends through the first and second extensions to define a pivot point, the second member pivoting relative to the first member about the pivot point to move the second member between the first and second configurations.

4. A surgical instrument as recited in claim 1, wherein the flange engages a lower surface of the second member when the second member is in the first configuration and the flange engages an upper surface of the second member when the second member is in the second configuration.

5. A surgical instrument as recited in claim 1, wherein the second portion of the first member comprises an upper surface and the second portion of the second member comprises a lower surface, the upper surface being spaced apart from the lower surface when the second member is in the first configuration and the upper surface engaging the lower surface when the second member is in the second configuration.

6. A surgical instrument as recited in claim 1, wherein a distal end surface of the first member is aligned with a distal end surface of the second member as the second member moves between the first and second configurations.

7. A surgical instrument as recited in claim 1, wherein the first extension comprises an inner surface defining a first channel and the second extension comprises an inner surface defining a second channel having at least a portion of the first extension disposed therein such that an outer surface of the first extension engages the inner surface of the second extension.

8. A surgical instrument as recited in claim 7, wherein:
the inner surfaces of the first and second extension define a lumen; and
the lumen has a maximum diameter when the second member is in the first configuration that is less than a maximum diameter of the lumen when the second member is in the second configuration.

9. A surgical instrument as recited in claim 1, wherein the first extension extends away from a bottom surface of the body and the projection extends away from a top surface of the body.

10. A surgical instrument as recited in claim 1, wherein the flange extends perpendicular to the second longitudinal axis.

11. A surgical instrument as recited in claim 1, wherein at least one of the first and second extensions comprises a light source.

12. A surgical instrument as recited in claim 11, further comprising a battery disposed in the second portion of the first member configured to provide power to the light source.

13. A surgical instrument as recited in claim 1, wherein the first and second members are each made entirely from molded plastic.

14. A surgical instrument as recited in claim 1, wherein:
the first extension comprises a first opening extending through a top surface of the first extension and a second opening extending through an inner surface of the first extension, the first and second openings being connected by a first passageway; and
the second extension comprises a third opening extending through a top surface of the second extension and a fourth opening extending through an inner surface of the second extension, the third and fourth openings being connected by a second passageway.

15. A surgical method comprising:
providing the surgical instrument of claim 1;
creating an incision;
creating a surgical pathway from the incision to a surgical site;
positioning the surgical instrument in the pathway with the second member in the first configuration; and
moving the second member from the first configuration to the second configuration to create a working channel.

16. A surgical method as recited in claim 15, wherein moving the second member from the first configuration to the second configuration comprises pivoting the second member relative to the first member.

17. A surgical method as recited in claim 15, wherein:
a pin extends through the first and second extensions to define a pivot point; and
moving the first member from the first configuration to the second configuration comprises pivoting the second member relative to the first member about the pivot point.

18. A surgical method as recited in claim 17, wherein the pin extends across a first channel defined by an inner surface of the first extension and across a second channel defined by an inner surface of the second extension.

19. A surgical method as recited in claim 15, wherein:
at least one of the first member and the second member include a light source; and
the method further comprises moving the light source from an off position to an on position to illuminate the working channel.

20. A surgical instrument, comprising:
a first member comprising a body including a first portion comprising a first extension defining a first longitudinal axis, the first extension comprising an inner surface defining a first channel, the body comprising a second portion including a projection defining a second longitudinal axis extending transverse to the first longitudinal axis, the projection comprising a flange;
a second member comprising a first portion including a second extension comprising an inner surface defining a second channel having at least a portion of the first extension disposed therein such that an outer surface of the first extension engages the inner surface of the second extension, the second member comprising a second portion comprising an opening, the projection extending through the opening, wherein at least one of the first and second extensions comprises a light source;
a pin extending through the first and second extensions to define a pivot point; and
a battery disposed in the second portion of the first member configured to provide power to the light source,
wherein the second member pivots about the pivot point between a first configuration in which the flange is positioned within the opening and the second extension extends parallel to the first longitudinal axis and a second configuration in which the flange is positioned outside of the opening and the second extension extends transverse to the first longitudinal axis,
wherein the flange engages a lower surface of the second member when the second member is in the first configuration and the flange engages an upper surface of the second member when the second member is in the second configuration,
wherein the first and second channels define a lumen having a maximum diameter when the second member is in the first configuration that is less than a maximum diameter of the lumen when the second member is in the second configuration, and
wherein the first and second members are each made entirely from molded plastic.
